(19) United States
(12) Patent Application Publication
(10) Pub. No.: US 2016/0199126 A1
(43) Pub. Date: Jul. 14, 2016

(54) BALLOON CATHETER ABLATION SYSTEM

(71) Applicant: JAPAN ELECTEL INC., Yokohama-shi, Kanagawa (JP)

(72) Inventor: Shutaro Satake, Kamakura-shi, Kanagawa (JP)

(21) Appl. No.: 14/759,647

(22) PCT Filed: Oct. 4, 2013
(86) PCT No.: PCT/JP2013/077042

§ 371 Date: Jul. 7, 2015

(51) Int. Cl. A61B 18/14; A61B 18/12

(52) U.S. Cl. CPC A61B 18/1492; A61B 18/1206; A61B 2018/00577

(57) ABSTRACT

A balloon catheter ablation system having a balloon (6) at a distal end of a catheter shaft (1). An electrode (11) for delivery of radiofrequency current and a temperature sensor (12) are arranged inside the balloon (6), Bipolar electrode pair (16a, 16b) are arranged on the outside of the balloon (6), with the balloon (6) sandwiched therebetween. When a fluid is injected into the inside of the balloon (6) by using a syringe (24), the balloon (6) expands and comes in close contact with a target site (S) of tissue. A vibration generator (25) agitates the fluid inside the balloon (6) by using vibration. A radiofrequency generator (31) conducts electricity between the electrode (11) for delivery of radiofrequency current and a counter electrode (13) and heats the fluid inside the balloon (6). Accordingly, the balloon (6) ablates the target site (S). The impedance between the bipolar electrode pair (16a, 16b) decreases as the ablation progresses. Accordingly, the progress of the ablation can be ascertained from the measurement results of an electrical impedance measuring potential amplifier (41).

### TECHNICAL FIELD

The present invention relates to a balloon catheter ablation system for heating and ablating a muscular tissue in contact with the surface of a balloon inflated in a lumen while supplying a radiofrequency power to electrodes inside the balloon.

### BACKGROUND ART

Atrial fibrillations often originate in the pulmonary vein, and many of these atrial fibrillations can be completely cured through pulmonary-vein isolation using a catheter ablation system. Such catheter ablation system is disclosed in, e.g., patent document 1.

According to an ordinary electrode catheter ablation, pulmonary vein potential is directly recorded using a diagnostic lasso electrode catheter to determine if the pulmonary vein has been isolated. As shown in FIG. 1, an ablation electrode 101 is brought into contact with the surface of a myocardial tissue located between a pulmonary vein (PV) and a left atrium to supply a radiofrequency current to the ablation electrode 101, thus heating and ablating a target site S on the myocardial tissue to thereby perform a pulmonary vein isolation. At the same time, the electric potential of the pulmonary vein (PV) is directly recorded while bringing the electrode portion of a diagnostic lasso electrode catheter 102, having a ring-shaped distal end, into close contact with the wall of the pulmonary vein. Also, according to such ordinary electrode catheter ablation, while a radiofrequency current is being delivered between a distal tip of the ablation electrode 101 and a counter electrode (not shown), there are monitored impedances therebetween and electric potentials from the distal tip of the ablation electrode 101, thus estimating the progress of ablation from changes in them.

On the other hand, according to a balloon catheter ablation, as shown in FIG. 2, pulmonary vein isolation is performed by inflating a balloon 104 inside the pulmonary vein (PV) and then supplying a radiofrequency current to an electrode 105 within the balloon 104 filled with a filling solution to thereby heat the whole of the balloon 104, thus heating and ablating a target site S on the myocardial tissue in contact with the balloon 104. At the same time, instead of a guide wire, a fine diagnostic lasso electrode catheter 107 is inserted into a catheter lumen 106 as an inner space of the catheter to bring its electrode into close contact with the wall of pulmonary vein, thereby directly recording the electric potential of the pulmonary vein (PV). For a further reference, see “diagnostic catheter to be inserted into a balloon catheter” (URL: http://www.medtronic.com/wcm/groups/mdtcom_sg/@mdt/@crdm/documents/images/contrib_152933.jpg;http://www.cryocath.com/en/images/home/arctic_front.jpg).

### PRIOR ART DOCUMENTS

### Patent Document

Patent Document 1: Japanese Unexamined Patent Application Publication No. 2002-126096

### SUMMARY OF THE INVENTION

### Problems to be Solved by the Invention

Manipulating the above-described diagnostic lasso electrode catheters (102,107) is technically difficult because each electrode thereof needs to be operated so as to come in close contact with the wall of the pulmonary vein in order to record its potentials. Also, referring to an example illustrated in FIG. 2, once the catheter lumen 106 is occupied with the diagnostic lasso electrode catheter 107, no guide wire can be used therein any longer, and therefore, an assist force against the balloon 104 gets so weak that the balloon cannot be brought into close contact with the ostium of the pulmonary vein, thus resulting in such problem that it becomes impossible to achieve the pulmonary vein isolation through one-shot ablation.

On the other hand, there was also devised another method in which an electrode for recording PV potentials is arranged at a distal end of the balloon catheter, which, however, has not yet been put into practical use since such catheter is bulky and bears a risk of thrombus adhesion.

Also, even if you monitor the impedance between the electrode 105 inside the balloon 104 and the counter electrode on a body surface as well as the electric potential of the electrode 105 inside the balloon 104, while delivering a radiofrequency current therebetween by means of balloon catheter ablation, they do not show sharp change therein due to being hindered by high impedance of the balloon membrane, thus making it impossible to estimate the progress of ablation.

In view of the problems described above, it is, therefore, an object of the present invention to provide a balloon catheter ablation system reflecting the progress of PV isolation, as an alternative to the conventional direct recording of pulmonary vein potential or the recording of electric impedance by means of a counter electrode and an electrode arranged inside the balloon.

### Means for Solving the Problems

In order to attain the above object, a first aspect of the present invention is a balloon catheter ablation system including:

a catheter shaft comprising an inner tube and an outer tube which are slidable to each other;

a balloon provided between distal ends of said inner tube and said outer tube, said balloon being elastic, having a high compliance;

an electrode for delivery of radiofrequency current and a temperature sensor, both being provided within said balloon;

a radiofrequency generator and a thermometer respectively connected to said electrode for delivery of radiofrequency current and said temperature sensor via a first electric wire;

a solution transport path defined by said outer tube and said inner tube, said solution transport path being in communication with an inside of said balloon;

a syringe for deflating and inflating said balloon and a vibration generator for agitating the inside of said balloon, said syringe and said vibration generator each connected to said solution transport path;

a bipolar electrode pair provided on the outside of said balloon, with said balloon sandwiched therebetween; and

an electric impedance measuring multimeter and a potential amplifier, each connected to said bipolar electrode pair via a second electric wire.

A second aspect of the present invention is a balloon catheter ablation system according to the first aspect, wherein said bipolar electrode pair is respectively arranged on a distal end of a balloon catheter, composed of said balloon and said catheter shaft, and a distal end of a guide sheath for inserting said balloon catheter therethrough, and are connected to said electric impedance measuring multimeter and said potential amplifier via said electric wire.

A third aspect of the present invention is a balloon catheter ablation system according to the first aspect wherein said bipolar electrode pair includes: a first balloon catheter anterior electrode arranged at a distal end of a balloon catheter, composed of said balloon and said catheter shaft; and a second balloon catheter anterior electrode placed behind said balloon, and are connected to said electric impedance measuring multimeter and said potential amplifier via said second electric wire.

A fourth aspect of the present invention is a balloon catheter ablation system according to the first aspect claim 1 wherein said bipolar electrode pair includes:

a first electrode attached to a guide wire, said guide wire being placed in front of said balloon, passing through a catheter lumen; and

any one of:
a second electrode arranged at a distal end of said outer tube;
a second electrode arranged at a distal end of a guide sheath for inserting therethrough a balloon catheter composed of said balloon and said catheter shaft; or
a second catheter anterior electrode placed behind said balloon, and

that said bipolar electrode pair is connected to said electric impedance measuring multimeter and said potential amplifier via said second electric wire.

A fifth aspect of the present invention is a balloon catheter ablation system according to any one of the first to fourth aspects, further including a radiofrequency noise cut filter attached to said electric impedance measuring multimeter and to said potential amplifier.

A sixth aspect of the present invention is a balloon catheter ablation system according to any one of the first to fifth aspects, wherein said bipolar electrode pair is formed of a metal having high electric conductivity and are cylindrically-shaped with a diameter of 3 mm or more and a length of 2 mm or more.

A seventh aspect of the present invention is a balloon catheter ablation system according to any one of the first to sixth aspects wherein said system is configured to measure an electric impedance across said balloon by means of said electric impedance measuring multimeter so as to measure the impedance that takes:

a low value reflecting an electric impedance of blood while said balloon is contracted; and then

an elevated value caused by the addition of a vascular impedance while said balloon is inflated within a blood vessel to completely block a flow of blood.

A eighth aspect of the present invention is a balloon catheter ablation system according to any one of the first to seventh aspects, wherein said system is configured to measure an electric impedance across said balloon by means of said electric impedance measuring multimeter so as to measure the impedance that takes:

a decreased value resulting from increased ion permeability of cell membranes while heating of a tissue through an ablation by said balloon is running smoothly; and then

an elevated value after the tissue gets vaporized or carbonized due to an excessive ablation.

A ninth aspect of the present invention is a balloon catheter ablation system according to any one of the first to eighth aspects further including a radiofrequency generator connectable across said bipolar electrode pair.

A tenth aspect of the present invention is a balloon catheter ablation system according to any one of the first to ninth aspects wherein said balloon is constituted using any one of: a cryoballoon; a laser balloon; an ultrasonic balloon; or a hot balloon employing a heating element or a nichrome wire as a heat source.

### Effects of the Invention

According to the first aspect of the present invention, the balloon, located at the distal end of the balloon catheter, is inflated by an electrolyte solution to come into close contact with an ostium of the pulmonary vein, followed by applying an electric current to the electrode for delivery of radiofrequency current, located inside the balloon, from the radiofrequency generator, thereby monitoring temperature of the balloon, electric impedance around the balloon, and far-field potentials, using the thermometer, the electric impedance measuring multimeter, and the potential amplifier, respectively, while agitating the inside of the balloon by the vibration generator.

Once the balloon is inserted into the blood vessel, an impedance across the balloon that is monitored by the electric impedance measuring multimeter, is allowed to reflect a blood impedance inside the blood vessel when the balloon is contracted, while when the balloon is inflated to block a blood flow inside the blood vessel, the vascular impedance is added to the blood impedance, causing an elevation in impedance. Once the ablation of balloon gets started, the blood vessel is heated to increase an ion permeability of cell membranes, thus reducing the impedance. Nevertheless, if the extent of ablation goes over the limit and the tissue gets vaporized or carbonized, the impedance will be elevated (FIG. 10).

Also, in a case where comparatively large and highly-conductive electrodes are employed as electrode pair, far-field potentials of a myocardial tissue can be recorded by the potential amplifier through a blood having a comparably high conductance, without bringing the electrodes into contact with the myocardial tissue. With the progress of ablation, potential intervals between left atrial and pulmonary vein get prolonged, decreasing the amplitude of the pulmonary vein potential recorded by the potential amplifier until it is finally vanished, thus signifying the achievement of pulmonary vein isolation.

Accordingly, in the atrial fibrillation ablation, there can be observed the progress of pulmonary vein isolation through the ablation by monitoring an electric impedance around the balloon and the far-field potentials, instead of directly recording the pulmonary vein potentials in a conventional manner.

According to the second aspect of the present invention, since only one bipolar electrode is provided on the balloon catheter, structure of the balloon catheter can be simplified.

According to the third aspect of the present invention, bipolar electrode(s) arranged on the balloon catheter is/are the balloon catheter anterior electrode only, thus simplifying the structure of the balloon catheter.

According to the fourth aspect of the present invention, an electrode arranged in front of the balloon catheter is attached not to a distal end of the balloon catheter but to the guide wire, thereby allowing the structure of the balloon catheter to be simplified.

According to the fifth aspect of the present invention, an electric impedance around the balloon and far-field potentials can be accurately measured by the electric impedance measuring multimeter and the potential amplifier without any interference of radiofrequency noise even during the application of a current to the electrode for delivery of radiofrequency current.

According to the sixth aspect of the present invention, contact areas between the bipolar electrode pair and the blood have a reduced impedance so that far-field potentials of cardiac muscle are allowed to be recorded by the potential amplifier through a blood even when the bipolar electrode and cardiac muscle are not directly in contact with each other.

According to the seventh aspect of the present invention, there can be observed a status of a vascular occlusion by way of the balloon, based on an output from the electric impedance measuring multimeter, such that one can infer the occurrence of a pinhole on the balloon if once-elevated electric impedance is observed to fall after blocking the blood vessel by the balloon.

According to the eighth aspect of the present invention, progress of the ablation of vascular tissue can be observed based on an output from the electric impedance measuring multimeter.

According to the ninth aspect of the present invention, if the surrounding ostium of the pulmonary vein cannot be completely ablated by the heating through thermal conduction from a hot balloon, a tissue surrounding the occlusion portion that remains to be ablated is enabled to undergo an additional ablation by directly applying a radiofrequency heating thereto through the application of a radiofrequency current from the bipolar electrode pair arranged across the balloon, with its blood vessel occluded by the balloon.

According to the tenth aspect of the present invention, measurement of electric impedance and far-field potential is applicable to any ablation system employing: a cryoballoon; a laser balloon; an ultrasonic balloon; or a hot balloon whose heat source is a heating element or a nichrome wire, thereby enabling the progress of ablation to be inferred.

### BRIEF DESCRIPTION OF THE DRAWING

FIG. 1 is an explanatory drawing illustrating a structure of a main body of a conventional electrode catheter ablation system.

FIG. 2 is an explanatory drawing illustrating a structure of a main body of a conventional balloon catheter ablation system.

FIG. 3 is an explanatory drawing illustrating a structure of a main body of a balloon catheter ablation system according to one embodiment of the present invention.

FIG. 4A is a drawing illustrating the conventional electrode catheter ablation system and a state of a blood flow and that of electric current before the ablation.

FIG. 4B is a drawing illustrating the balloon catheter ablation system according to the embodiment shown in FIG. 3 and a state of a blood flow and that of electric current before the ablation.

FIG. 5A is a drawing illustrating the conventional electrode catheter ablation system and a state of a blood flow and that of electric current after the ablation.

FIG. 5B is a drawing illustrating the balloon catheter ablation system according to the embodiment shown in FIG. 3 and a state of a blood flow and that of electric current after the ablation.

FIG. 6 is an explanatory drawing illustrating a structure of a main body of a balloon catheter ablation system according to another modified embodiment of the present invention.

FIG. 7 is an explanatory drawing illustrating a structure of a main body of a balloon catheter ablation system according to another modified embodiment of the present invention.

FIG. 8 is an explanatory drawing illustrating a structure of a main body of a balloon catheter ablation system according to another modified embodiment of the present invention.

FIG. 9 is a diagram of the balloon catheter of the present invention in use.

FIG. 10 is a diagram illustrating the relationship between a balloon temperature, an electric impedance around the balloon and an amplitude of far-field potential in the course of balloon ablation of the present invention.

### MODE FOR CARRYING OUT THE INVENTION

As follows is a detailed description of embodiments of a balloon catheter ablation system provided by the present invention with reference to the appended drawings.

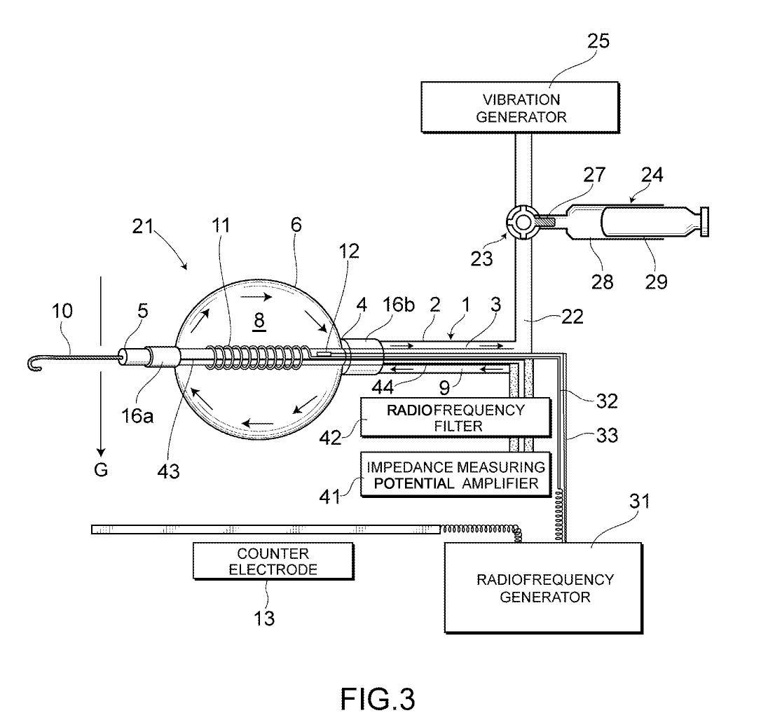

FIG. 3 shows a structure of a main body of a balloon catheter ablation system according to one embodiment of the present invention. In FIG. 3, numeral 1 denotes a cylindrical catheter shaft which is insertable into a luminal organ and is richly flexible, and comprises an outer tube 2 and an inner tube 3 which are slidable with each other in a longitudinal direction. An inflatable/contractable balloon 6 is provided between a distal end 4 of the outer tube 2 and a vicinity of a distal end 5 of the inner tube 3. The balloon 6 is made up of a resin rich in heat resistance, such as polyurethane, PET (polyethylene terephthalate) or the like, and formed in a thin membrane form. The balloon 6 is inflated to take a shape of a rotating body, for example, an approximately spherical shape by filling an inside of the balloon 6 with a solution (normally, a mixture of a physiological saline and a contrast medium).

A solution transport path 9 is formed between the outer tube 2 and the inner tube 3 inside the catheter shaft 1, to transport a solution to this filling part 8 and transmit vibrational waves thereto, in communication with the filling part 8 formed inside the balloon 6. Numeral symbol 10 denotes a guide wire for guiding the balloon part 8 to a target site and this guide wire 10 is provided through the inner tube 3.

An electrode 11 for delivery of radiofrequency current and a temperature sensor 12 are each arranged inside the balloon 6. The electrode 11 for delivery of radiofrequency current is arranged in such a coiled fashion that it is wound around the inner tube 3 in order to heat the inside of the balloon 6. Further, the electrode 11 for delivery of radiofrequency current is of a monopolar structure, and is able to conduct a radiofrequency current between itself and a counter electrode 13 provided on the outside the catheter shaft 1. Then, the electrode 11 for delivery of radiofrequency current generates heat by applying a radiofrequency current thereto. Alternatively, the electrode 11 for delivery of radiofrequency current may be bipolar to apply a radiofrequency current across both electrodes.

A temperature sensor 12 as a temperature detection part is provided on the proximal end side of the inner tube 3 inside the balloon 6, and is configured to contact the electrode 11 for delivery of radiofrequency current to detect the temperature thereof. In the meantime, though not shown in the figures in the present embodiment, another temperature sensor for detecting the inside temperature of the balloon 6 may be fixed to the vicinity of the distal end 5 of the inner tube 3 in addition to the temperature sensor 12.

Further, on the outside the balloon 6, the bipolar electrode pair 16a, 16b are respectively provided on a vicinity of a distal end 5 of the inner tube 3 and a distal end 4 of the outer tube 2 with the balloon 6 being sandwiched therebetween. According to the embodiment illustrated in FIG. 3, it is configured such that a current is applied between one electrode 16a and the other electrode 16b with a blood flow flowing in the luminal organ being completely blocked by allowing the balloon 6 to be closely attached to a wall surface of the luminal organ. Then, the catheter shaft 1 and the balloon 6 make up the balloon catheter 21, with a shape insertable into the body.

On the outside of the balloon catheter 21, a solution transport pipe 22 is connected in communication with a proximal end of the solution transport path 9. Two ports of a three-way cock 23 are coupled to a middle of this solution transport pipe 22, and the remaining one port of the three-way cock 23 is coupled to a syringe 24 for deflating and inflating the balloon 6. Further, a vibration generator 25 for agitating the inside of the balloon 6 is connected to a proximal end of the solution transport pipe 22. The three-way cock 23 has an operation piece 27 capable of being pivotally operated by the fingers such that the solution transport path 9 may be connected in communication with either the syringe 24 or the vibration generator 25 by operating the operation piece 27.

A syringe 24 as a solution injector comprises a cylindrical body 28 connected to the three-way cock 23a and a movable plunger 29 within the same. If the plunger 29 is pushed with the syringe 24 being communicated with the solution transport path 9 by the three-way cock 23, the solution is supplied from the inside of the cylindrical body 28 into the inside of the balloon 6 via the solution transport path 9, while if the plunger 29 is pulled, the solution is recovered from the inside of the balloon 6 into the inside of the cylindrical body 28 via the solution transport path 9.

The vibration generator 25, making up an intra-balloon agitating unit along with the solution transport pipe 22, applies asymmetric vibrational waves to the solution inside the balloon 6 through the solution transport path 9, with the vibration generator 25 being communicated with the solution transport path 9 by the three-way cock 23, to thereby steadily generate swirls thereinside. The solution inside the balloon 6 is vibrated and agitated by the swirls inside the balloon 6 to keep the temperature inside the balloon 6 uniform.

Further, a radiofrequency generator 31 is provided outside of the balloon catheter 21, and the electrode 11 for delivery of radiofrequency current and the temperature sensor 12 placed inside the balloon 6 are electrically connected to the radiofrequency generator 31 through electric wires 32, 33 placed inside the catheter shaft 1, respectively. The radiofrequency generator 31 supplies a radiofrequency energy, being an electric power, to between the electrode 11 for delivery of radiofrequency current and the counter electrode 13 through the electric wire 32 and heats the whole of the balloon 6 filled with the solution, and is provided with a thermometer (not shown) for measuring and outputting the temperature of the electrode 11 for delivery of radiofrequency current, and eventually, the inside temperature of the balloon 6 by a detection signal from the temperature sensor 12 transmitted through the other electric wire 33 and then displaying such temperature. Further, the radiofrequency generator 31 is configured to sequentially take in information on temperatures measured by the thermometer to determine radiofrequency energy to be supplied to between the electrode 11 for delivery of radiofrequency current and the counter electrode 13 through electric wire 32. Note that the electric wires 32, 33 are fixed along the inner tube 3 over the entire axial length of the inner tube 3.

Whilst the electrode 11 for delivery of radiofrequency current is used as a heating means for heating the inside of the balloon 6 in the present embodiment, it is not limited to any specific ones as long as it is capable of heating the inside of the balloon 6. For example, as substitute for the electrode 11 for delivery of radiofrequency current and the radiofrequency generator 31, there may be employed any one of couples of: an ultrasonic heating element and ultrasonic generator; a laser heating element and laser generator; a diode heating element and diode power supply; or a nichrome wire heating element and nichrome wire power supply unit.

Further, the balloon catheter comprising the catheter shaft 1 and the balloon 6 is made of such a heat resistant resin that can withstand heating without causing thermal deformation and the like when heating the inside of the balloon 6. As for the shape of the balloon 6, except the spherical shape whose long and short axes are equal, it may take various shapes of rotational bodies such as an oblate spherical shape whose short axis is defined as a rotation axis, a prolate spheroid whose long axis is defined as a rotation axis, or a bale shape. In any of these shapes, the balloon is made up of such a high-compliance elastic member that deforms when it comes in close contact with an inside wall of a luminal organ.

Further, according to the present embodiment, an electric impedance measuring potential amplifier 41 and a radiofrequency filter 42 are each arranged outside the balloon catheter 21. The electric impedance measuring potential amplifier 41 is connected to the electrodes 16a, 16b arranged at the front and rear of the balloon 6 via the electric wires 43, 44, respectively, allowing a weak current to flow between the bipolar electrode pair 16a, 16b, measuring an electric impedance obtained from the potential value at that time, and outputting the same as an electric impedance surrounding the balloon 6; and further, amplifying a far-field potential obtained from the bipolar electrode pair 16a, 16b, and recording and outputting the same, and determining whether or not the ablation effect, eventually, pulmonary vein isolation has been successfully achieved, based on the changes in electric impedance and potential waveform. Also, the radiofrequency noise cut filter 42 is incorporated into the electric circuit for measurement composed of the bipolar electrode pair 16a, 16b, the electric impedance measuring potential amplifier 41 and the electric wires 43, 44 in order to eliminate the influence by the radiofrequency noise generated from the radiofrequency generator 31. In the same way as the foregoing electric wires 32, 33, the electric wires 43, 44 are fixed along the inner tube 3 over the entire axial length of the inner tube 3.

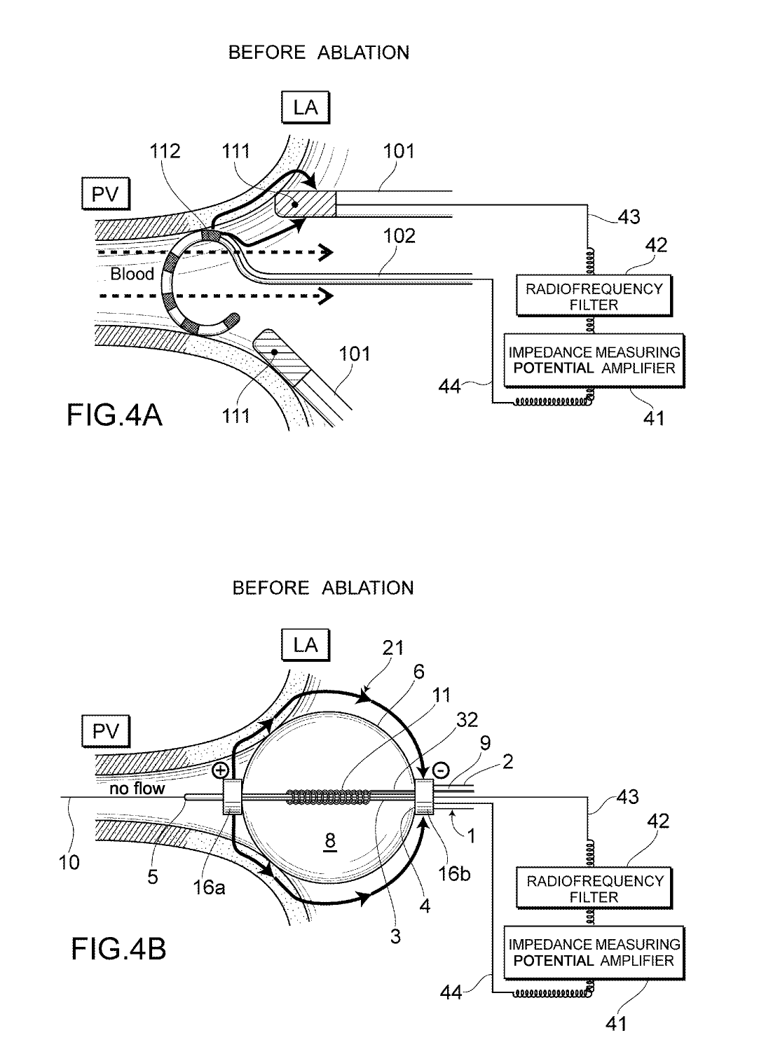

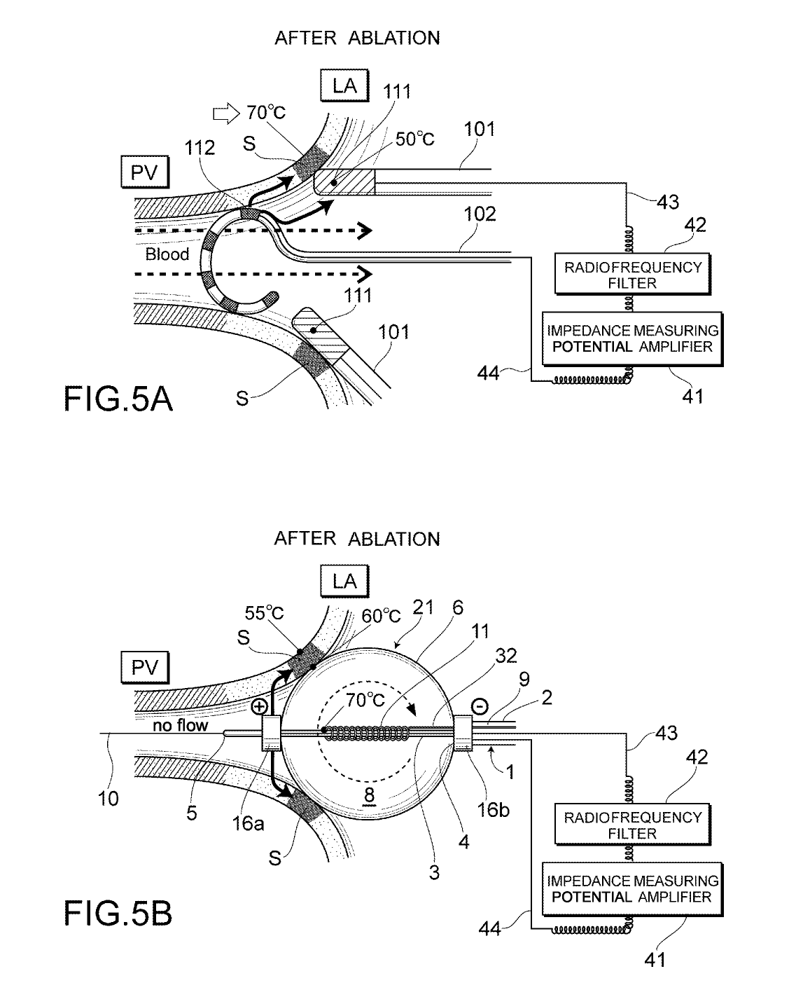

Next is a description of the operating principle of the balloon catheter ablation system according to the present embodiment with reference to FIGS. 4 and 5, in which FIG. 4 shows the state of blood flow and electric current before ablation, while FIG. 5 shows that after ablation. In any of FIGS. 4 and 5, blood flow is illustrated by a thick dotted arrow and electric current by a thick solid arrow. Further, FIGS. 4A and 5A show a conventional electrode catheter ablation system in which an electrode part 111 of an ablation electrode 101 and an electrode part 112 of a diagnostic lasso electrode catheter 102 are coupled to an electric impedance measuring potential amplifier 41 including a radiofrequency filter 42 via electric wires 43, 44. In contrast, FIGS. 4B and 5B show a balloon catheter ablation system of the present embodiment shown in FIG. 3 described above.

In these figures, when the opening of a pulmonary vein is circumferentially and transmurally ablated by heat from the ablation electrode 101 or the balloon 6, then the electric impedance between a myocardial sleeve in the pulmonary vein PV and a left atrium LA is changed. According to the ordinary ablation method shown in FIGS. 4A and 5A, measurement of the electric impedance between the electrode part 111 of the ablation electrode 101 and the electrode part 112 of the diagnostic lasso electrode catheter 102 indicates that the electric impedance measured by the electric impedance measuring potential amplifier 41 remains unchanged before and after the ablation because an electric current is allowed to flow in a blood having a low resistance due to no blood flow in the pulmonary vein PV being blocked even when the conduction in the cardiac muscle is lost.

In the balloon catheter 21 shown in FIGS. 4B and 5B, however, the impedance across the balloon 6 has a low value, reflecting the impedance of the blood in the vein when the balloon 6 is contracted, while it becomes elevated due to the impedance of a vascular tissue being added thereto when the blood flow in the pulmonary vein PV is completely blocked by inflating the elastic balloon 6. At this time, if the vascular tissue at the target site S between the myocardial sleeve in the pulmonary vein and the left atrium LA is heated by the ablation, then the ion permeability of the cell membrane is enhanced, and thus the electric impedance across the balloon 6 drops. Accordingly, if the electric impedance measured by the electric impedance measuring potential amplifier 41 drops when the radiofrequency generator 31 measures and displays the inside temperature of the balloon 6 by taking in a detection signal from the temperature sensor 12, indicating that the inside temperature of the balloon 6 reaches a given target temperature, then, the ablation at the target site S by the balloon catheter 21 can be determined to be going smoothly by the electric impedance measuring potential amplifier 41. If, however, vaporization or carbonization of the tissue occurs due to excessive ablation, then the electric impedance turns upward. At this time, from a measurement output of the electric impedance by the electric impedance measuring potential amplifier 41, energization of the electrode for delivery of radiofrequency current 11 should be immediately discontinued. As for the far-field potentials captured by the bipolar electrode pair 16a, 16b and recorded by the electric impedance measuring potential amplifier 41, with the progress of ablation, the potential interval between the left atrium LA and the pulmonary vein PV gets prolonged, and the pulmonary vein potential decreases its amplitude, eventually to zero, when pulmonary vein isolation has been successfully achieved.

Next is a description of various modified embodiments of the above balloon catheter ablation system.

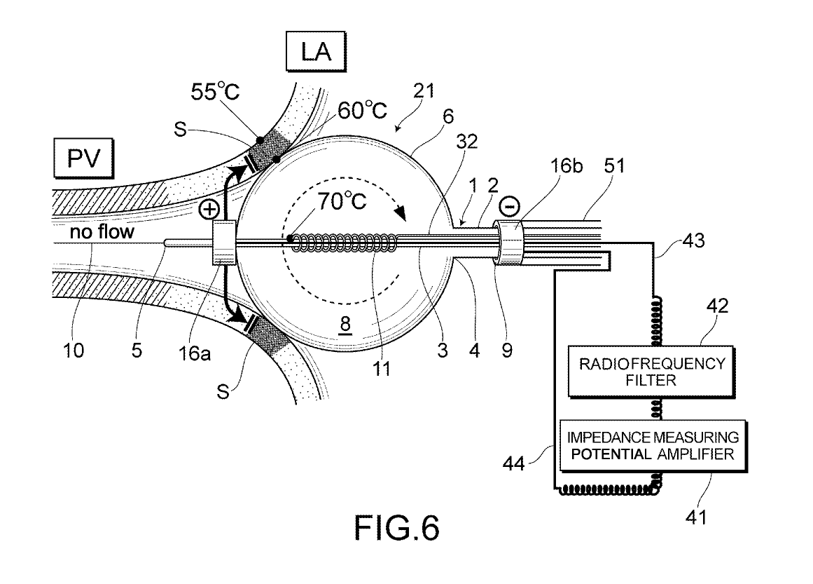

According to a first modified embodiment shown in FIG. 6, on the outside of the balloon 6 is arranged the electrode 16a, i.e., one of the electrodes constituting the bipolar electrode pair, at the distal end 5 of the inner tube 3 which is also a distal end of the balloon catheter 21, while the other electrode 16b is arranged at a distal end of a cylindrical guide sheath 51 for inserting the balloon catheter 21 therethrough. The electric impedance measuring potential amplifier 41 is connected to the electrodes 16a, 16b via the electric wires 43, 44, respectively. In this case, the bipolar electrode arranged in the balloon catheter 21 is only one electrode 16a, thus enabling the structure of the balloon catheter 21 to be simplified. Meanwhile, the guide sheath 51 is used to insert the balloon catheter 21 into the pulmonary vein PV in the body in the foregoing embodiments and herein-below described modified embodiments.

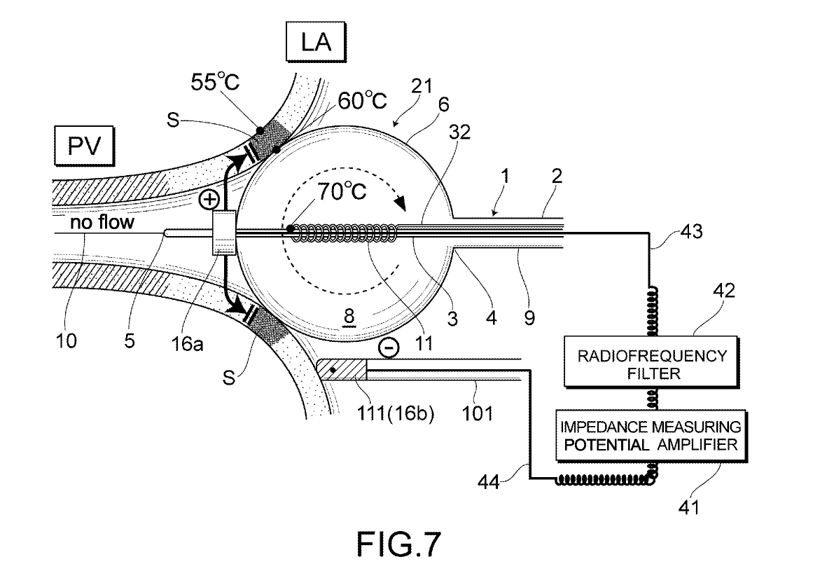

According to a second modified embodiment shown in FIG. 7, on the outside of the balloon 6 is arranged one electrode 16a, i.e., one of the electrodes constituting the bipolar electrode pair, at a distal end 5 of the inner tube 3 which is a distal end of balloon catheter 21, while the other electrode 16b, as another catheter anterior electrode placed in the rear of the balloon 6, is arranged at a distal end of the ablation electrode 101. That is, the electrode 16b given here corresponds to the electrode part 111 of the aforementioned ablation electrode 101. The electric impedance measuring potential amplifier 41 is connected to the electrodes 16a, 16b via the electric wires 43, 44, respectively. In this case also, the bipolar electrode arranged in the balloon catheter 21 is only one electrode 16a or the balloon catheter anterior electrode, and the structure of the balloon catheter 21 can be simplified.

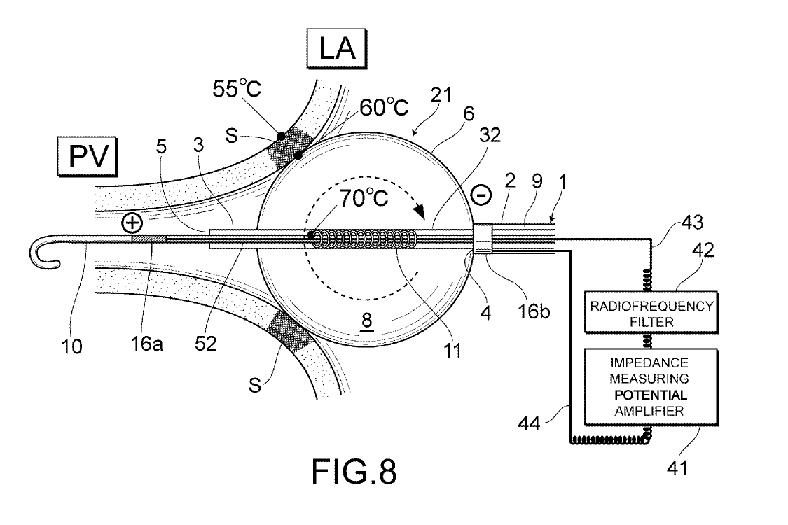

According to a third modified embodiment shown in FIG. 8, on the outside of the balloon 6 is arranged one electrode 16a, i.e., one of the electrodes constituting the bipolar electrode pair, at the distal portion of the guide wire 10, and the other electrode 16b is arranged at the distal end 4 of the outer tube 2. The guide wire 10, passing through the catheter lumen 52 or an internal space of the inner tube 3, has the distal end thereof located in front of the balloon 6. The electrode 16a, as an electrode attached to the guide wire, is placed in the distal end of the guide wire 10. The electric impedance measuring potential amplifier 41 is connected to the electrodes 16a, 16b via the electric wires 43, 44, respectively.

The electrode 16b may be located at a distal end of the guide sheath 51 as shown in the first modified embodiment, or located at a distal end of the ablation electrode 101 as the other catheter anterior electrode placed in the rear of the balloon 6 as shown in the second modified embodiment. In either case, the bipolar electrode arranged in the balloon catheter 21 is only one electrode 16a which is an electrode attached to the guide wire, and thus the structure of the balloon catheter 21 can be simplified.

In each of the embodiments illustrated in FIGS. 3 and 6 to 8, the electrode 16a, as one of the bipolar electrode pair, is arranged anterior to a close-contact part between the inflated balloon 6 and an inside wall of a luminal organ, while the other electrode 16b thereof is arranged posterior to the close-contact part. At this time, if a blood flow between the front and rear parts of the close-contact part is completely blocked, progress of ablation can be accurately determined by monitoring the change of electric impedance around the balloon 6, using the electric impedance measuring potential amplifier 41. Also, the bipolar electrode pair 16a, 16b may preferably be made of a metal such as gold, silver or copper with high conductivity, having a cylindrical shape with a diameter of 3 mm or more and a length of 2 mm or more. Thus, the contact area with the blood in the pulmonary vein can be increased, so that the electric impedance is decreased to increase the conductivity, enabling the far-field potential to be easily detected. Further, due to the shape free from irregularities, adhesion of a blood clot can be prevented.

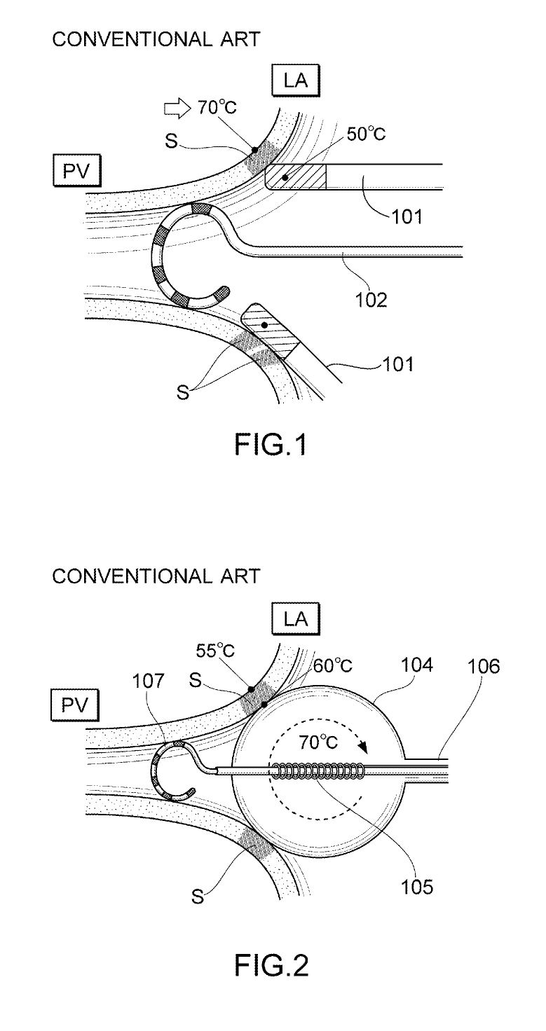

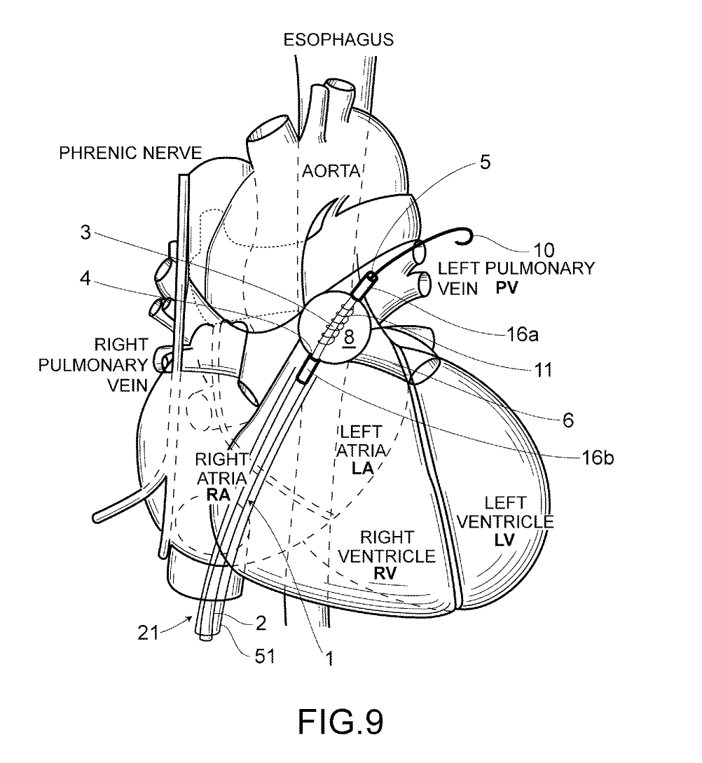

Next is a description of how the balloon catheter ablation system according to the present invention is actually used with reference to FIG. 9 that shows the balloon catheter 21 of FIG. 1 in use.

The guide wire 10 is inserted into the left atrium LA by making a puncture in an interatrial septum through a femoral vein, and then the guide sheath 51 is placed in the left atrium LA through the guide wire 10, allowing the balloon catheter 21 to be inserted into the pulmonary vein PV through the guide sheath 51. With the support by the guide wire 10 and the guide sheath 51, the inside of a high-compliance elastic balloon 6 is inflated by injecting thereinto a mixed solution of physiologic saline and an ionic contrast medium, thereby bringing the same into close contact with the orifice of a pulmonary vein. This is checked by injecting a contrast medium from the distal end of the catheter and obtaining an image of the pulmonary vein blocked. At this time, the blood flow in the pulmonary vein PV and the left atrium LA is completely blocked by the balloon 6 inflated.

The temperature and energizing time of the balloon 6 is determined in accordance with the development level of a myocardial sleeve measured by CT (computed tomography), and then the radiofrequency generator 31 acting as a device for delivery of radiofrequency current and the vibration generator 25 for agitating the inside of the balloon 6 are switched on, thereby initiating the ablation of the target site S closely contacted by the balloon 6, by performing the conduction of a radiofrequency current between the electrode 11 for delivery of radiofrequency current and the counter electrode 13, while monitoring the temperature of the inside of the balloon 6 by the temperature sensor 12. At the same time, an electric impedance around the balloon 6 and far-field potential are monitored and output by the external bipolar electrode pair 16a, 16b in the front and rear of the balloon 6, using the electric impedance measuring potential amplifier 41. If the inside temperature of the balloon 6 reaches a desired target temperature, and the electric impedance around the balloon 6 is decreased with a certain change in potential waveform being observed, then it indicates that the ablation around the orifice of the pulmonary vein is proceeding smoothly, and thus the energization is to be continued. On the other hand, if the electric impedance around the balloon 6 is not decreased or potential waveforms remain unchanged even after the inside temperature of the balloon 6 reaches a target temperature, there is a high possibility that the energization may be ineffectively performed, and thus the energization to the electrode 11 for delivery of radiofrequency current is to be stopped, and the energization is to be tried again after changing the position of the balloon 6. This way, when the desired ablation has been accomplished and the pulmonary vein PV potential has vanished, the balloon catheter 21 is to be removed.

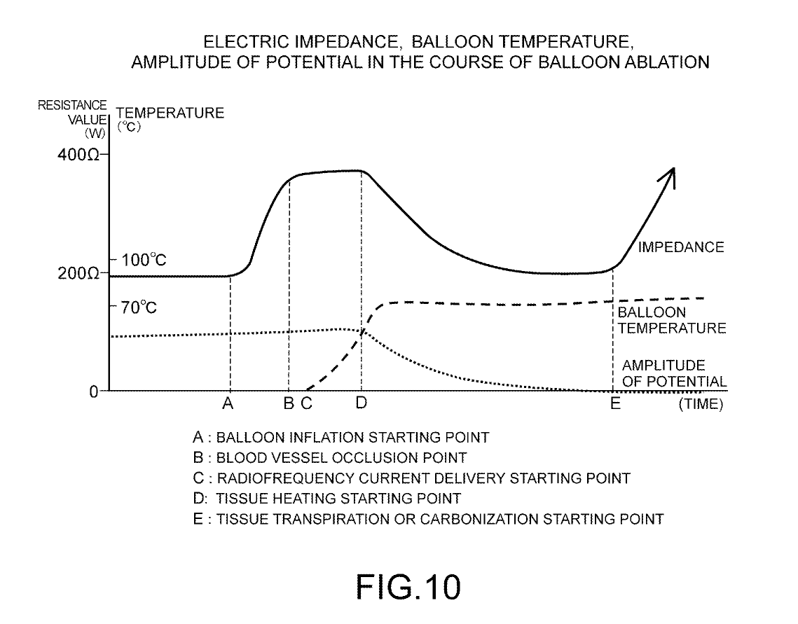

FIG. 10 shows a temperature inside the balloon 6 monitored by the thermometer of the radiofrequency generator 31 during the delivery of radiofrequency current to the electrode 11 as “balloon temperature”; and an electric impedance and an amplitude of a pulmonary vein far-field potential around the balloon 6 monitored by the electric impedance measuring potential amplifier 41 during that time as “impedance” and “potential amplitude”, respectively. In the same Figure, A: when the inflation of balloon 6 is started, increase of the electric impedance is observed, B: when the blood vessel is completely blocked by the balloon 6, the electric impedance reaches a maximum. C: when the delivery of radiofrequency current to the target site S by the balloon catheter 21 is started, D: heating a tissue gets started as the inside temperature of the balloon 6 rises, and the decrease of the electric impedance around the balloon 6 is observed. Also, the amplitude of pulmonary vein far-field potential is decreased and finally reduced to zero. This indicates that the pulmonary vein isolation has been successfully achieved by the ablation. If the ablation is excessively continued, E: the tissue gets vaporized or carbonized, and the impedance then gets elevated.

To summarize the above, the balloon catheter ablation system according to the present invention comprises: the catheter shaft 1 comprising the inner tube 3 and the outer tube 2 which are slidable to each other; the balloon 6 provided at the distal portion of the balloon catheter 21 between distal ends of the inner tube 3 and the outer tube 2; the electrode 11 for delivery of radiofrequency current and the temperature sensor 12, both being provided within the balloon; the radiofrequency generator 31 outside the body with the thermometer incorporated thereinto, respectively connected to the electrode 11 and the temperature sensor 12 via the electric wires 32, 33 acting as first electric wires within the catheter shaft 1; the solution transport path 9 defined by the outer shaft 2 and the inner shaft 3, in communication with the inside of the balloon 6; the syringe 24 for deflating and inflating the balloon, and the vibration generator 25 for agitating the inside of the balloon, the syringe 24 and the vibration generator 25 each connected to the solution transport path 9; the bipolar electrode pair 16a, 16b provided outside the balloon 6 with the balloon 6 sandwiched therebetween; and the electric impedance measuring potential amplifier 41 outside the body, constituting the electric impedance measuring multimeter and the potential amplifier, connected to the bipolar electrode pair 16a, 16b via the electric wires 43, 44 serving as second electric wires.

In this case, in order to apply the balloon catheter ablation system of the present invention to pulmonary vein isolation for treatment of atrial fibrillation, the balloon, located at the distal end of the balloon catheter 21, is inflated by an electrolyte solution to let the balloon come into close contact with an ostium of the pulmonary vein, followed by applying the electric current to the electrode 11 for delivery of radiofrequency current, located inside the balloon, from the radiofrequency generator 31, while agitating the inside of the balloon by the vibration generator 25, monitoring the temperature of the balloon 6, the electric impedance around the balloon 6 and far-field potentials by the thermometer of the radiofrequency generator 31 and the electric impedance measuring potential amplifier 41, respectively.

Here, once the balloon has been inserted into the blood vessel, the impedance across the balloon 6 monitored by the electric impedance measuring potential amplifier 41, reflects a blood impedance inside the blood vessel when the balloon 6 is contracted, while when the balloon 6 is inflated to block the blood flow inside the blood vessel, then the vascular impedance is added to the blood impedance, causing an elevation in impedance. Once the ablation by the balloon gets started, the blood vessel is to be heated to increase an ion permeability of cell membranes, thus reducing the impedance. Nevertheless, if the extent of ablation goes over the limit and the tissue gets vaporized or carbonized, the impedance will be elevated again.

Moreover, if large and highly-conductive electrodes are employed as the electrode pair 16a, 16b, then the far-field potentials of a myocardial tissue can be recorded by the potential amplifier of the electric impedance measuring potential amplifier 41 through a blood, which is comparably highly conductive, without bringing the electrodes 16a, 16b into close contact with the myocardial tissue. With the progress of ablation, potential intervals between the left atrial and the pulmonary vein get prolonged, and the pulmonary vein potential, recorded by the potential amplifier of the electric impedance measuring potential amplifier 41, decreases its amplitude to be vanished, thus signifying the achievement of pulmonary vein isolation.

As discussed above, as the decrease of electric impedance and the change in potential waveform, associated with the balloon 6 having reached the target temperature, reflect the progress of pulmonary vein isolation, one can observe effects of the pulmonary vein isolation by means of hot balloon ablation, from the electric impedance around the balloon 6 and the monitor output of the potential waveform, thus providing barometers for determining whether the energization was/is effective or not. Accordingly, progress of pulmonary vein isolation by ablation can be observed by monitoring an electric impedance across the balloon 6 and the far-field potentials, using the electric impedance measuring potential amplifier 41, instead of directly recording the pulmonary vein potentials in a conventional manner.

Further, as shown in FIG. 6, the bipolar electrode pair 16a, 16b are respectively arranged on the distal end of the balloon catheter 21 composed of the balloon 6 and the catheter shaft 1, and the distal end of the guide sheath 51 for inserting therethrough the balloon catheter 21 and are connected to the electric impedance measuring potential amplifier 41 via the electric wires 43, 44.

In this case, since only one electrode out of the bipolar electrode pair is provided on the balloon catheter 21, structure of the balloon catheter 21 can be simplified.

Alternatively, as shown in FIG. 7, the bipolar electrode pair may include a first balloon catheter anterior electrode 16a arranged at a distal end of the balloon catheter 21; and a second balloon catheter anterior electrode 16a placed behind the balloon 6, and are connected to the electric impedance measuring potential amplifier 41 via the electric wires 43, 44.

In this case, since only one electrode 16a out of the bipolar electrode pair, as the balloon catheter anterior electrode, is provided on the balloon catheter 21, structure of the balloon catheter 21 can be simplified as well.

Still alternatively, as shown in FIG. 8, the bipolar electrode pair 16a, 16b may include: a first electrode 16a attached to the guide wire 10 placed in front of the balloon 6 through the catheter lumen 52; and any one of: a second electrode arranged at the distal end 4 of the outer tube 2; a second electrode arranged at the distal end of the guide sheath 51 for inserting therethrough the balloon catheter 21; or a second catheter anterior electrode placed behind the balloon 6, as a second electrode 16b, and that the bipolar electrode pair is connected to the electric impedance measuring potential amplifier 41 via the electric wires 43,44.

In this case, the electrode 16a arranged in front of the balloon catheter 21 is arranged not on a distal end of the balloon catheter 21 but attached to the guide wire, thereby enabling the structure of the balloon catheter 21 to be simplified.

Further, the electric impedance measuring potential amplifier 41 of the present embodiment has the radiofrequency filter 42 acting as a radiofrequency noise cut filter, as attached thereto.

In this case, an electric impedance around the balloon and far-field potentials can be accurately measured by the electric impedance measuring potential amplifier 41 without any interference by radiofrequency noises even during the application of a current to the electrode 11 for delivery of radiofrequency current.

Further, since the bipolar electrode pair 16a, 16b of the present embodiment are each formed of a metal having high electric conductivity, and has a comparatively large cylindrical shape with a diameter of 3 mm or more and a length of 2 mm or more, contact area between the cylindrically-shaped bipolar electrode pair 16a, 16b and the blood of pulmonary vein is enlarged to reduce the impedance of the contact area between the bipolar electrode pair 16a, 16b and the blood, enhancing the conductivity therebetween so that the far-field potentials at the cardiac muscle are allowed to be recorded by the electric impedance measuring potential amplifier 41 through blood even when the bipolar electrode and cardiac muscle are not in direct contact with each other. Moreover, since the bipolar electrode pair 16a, 16b are shaped so as to be free from irregularities, thrombus adhesion can be prevented.

Further, according to the present embodiment, the electric impedance measuring potential amplifier 41 is configured to measure an electric impedance across the balloon 6 and output its result to, e.g., a display by means of the electric impedance measuring potential amplifier 41 so as to measure the impedance that takes: a low value reflecting an electric impedance of blood while the balloon 6 is contracted; or an elevated value caused by the addition of a vascular impedance while the balloon is inflated within a blood vessel to completely block a flow of blood inside the blood vessel.

In this case, there can be observed an occlusion status, by the balloon, based on an output from the electric impedance measuring potential amplifier 41; one can infer the presence of a pinhole occurrence if once-elevated electric impedance is observed to have fallen after the blood vessel has been blocked by the balloon 6.

Also, according to the present embodiment, electric impedance measuring potential amplifier 41 is configured to measure an electric impedance across the balloon 6 and output its result to, e.g., a display by means of the electric impedance measuring potential amplifier 41 so as to measure the impedance that takes: a decreased value resulting from increased ion permeability of cell membranes while heating of a tissue through an ablation by the balloon 6 is running smoothly; and then an elevated value after the tissue gets vaporized or carbonized due to an excessive ablation.

In this case, progress of ablation of a vascular tissue can be observed based on an output from the electric impedance measuring potential amplifier 41.

Alternatively, in the present embodiment, the radiofrequency generator 31 may be configured so as to be connectable between the bipolar electrode pair 16a, 16b. Such configuration, for example, can be achieved by providing a switch for allowing the bipolar electrode pair 16a, 16b to be connected either to the electric impedance measuring potential amplifier 41 or to the radiofrequency generator 31.

In this case, if the surrounding ostium of the pulmonary vein could not be perfectly ablated by the heating of thermal conduction from the hot balloon 6, there can be performed an additional ablation by directly applying a radiofrequency heating to a remaining tissue surrounding the occlusion portion, through the application of a radiofrequency current from between the bipolar electrode pair 16a, 16b arranged across the balloon 6, with its blood vessel being blocked by the balloon 6.

Also, the balloon 6 of the present invention may be constituted using any one of: a cryoballoon; a laser balloon; an ultrasonic balloon; or a hot balloon employing a heating element or a nichrome wire as a heat source.

In this case, measurement of an electric impedance and that of far-field potential is applicable to a system employing: a cryoballoon; a laser balloon; an ultrasonic balloon; or a hot balloon which employs a heating element or a nichrome wire as a heat source, thereby enabling the progress of ablation to be inferred.

In addition to the foregoing, as an effect specific to the embodiment, the balloon catheter 21, including the catheter shaft 1 and the balloon 6, is entirely made of a heat-resisting material.

In this case, thermal deformations of the balloon catheter 21 including the balloon 6 can be prevented when heating the inside of the balloon 6 during the energization of the electrode 11 for delivery of radiofrequency current.

In addition, the present invention is not limited to the foregoing embodiments and various modifications are possible within the scope of the gist of the present invention. Respective configurations of the catheter 21 shaft 11 or the balloon 6 are not limited to those described above and various ones may be applicable in accordance with the target sites. Although there is illustrated such configuration that the thermometer is integrated into the radiofrequency generator 31 in the foregoing embodiments, the radiofrequency generator 31 and the thermometer may be separately arranged.

### DESCRIPTION OF THE REFERENCE SYMBOLS

1 catheter shaft
2 outer shaft (outer tube)
3 inner shaft (inner tube)
6 balloon
9 solution transport path
10 guide wire (wire)
11 electrode for delivery of radiofrequency current
12 temperature sensor
16a bipolar electrode (balloon catheter anterior electrode)
16b bipolar electrode (posterior electrode)
21 balloon catheter
24 syringe (syringe for deflating and inflating balloon)
25 vibration generator
31 radiofrequency generator (thermometer)
41 electric resistance measuring apparatus (current resistance measuring multimeter)
42 radiofrequency filter (radiofrequency noise cut filter)
32,33 electric wire (first electric wire)
43,44 electric wire (second electric wire)
51 guide sheath

1. A balloon catheter ablation system comprising:
a catheter shaft comprising an inner tube and an outer tube which are slidable to each other;
a balloon provided between distal ends of said inner tube and said outer tube said balloon being elastic;
an electrode for delivery of radiofrequency current and a temperature sensor, both being provided within said balloon;
a radiofrequency generator and a thermometer respectively connected to said electrode for delivery of radiofrequency current and said temperature sensor via a first electric wire;
a solution transport path defined by said outer tube and said inner tube, said solution transport path being in communication with an inside of said balloon;
a syringe for deflating and inflating said balloon and a vibration generator for agitating the inside of said balloon, said syringe and said vibration generator each connected to said solution transport path;
a bipolar electrode pair provided on the outside of said balloon, with said balloon sandwiched therebetween; and
an electric impedance measuring multimeter and a potential amplifier, each connected to said bipolar electrode pair via a second electric wire,
wherein said bipolar electrode pair includes: a first balloon catheter anterior electrode arranged at a distal end of a balloon catheter, composed of said balloon and said catheter shaft; and a second balloon catheter anterior electrode placed behind said balloon.

2. (canceled)

3. (canceled)

4. (canceled)

5. The balloon catheter ablation system according to claim 1, further comprising a radiofrequency noise cut filter attached to said electric impedance measuring multimeter and to said potential amplifier.

6. The balloon catheter ablation system according to claim 1 wherein said bipolar electrode pair is cylindrically-shaped with a diameter of 3 mm or more and a length of 2 mm or more.

7. The balloon catheter ablation system according to claim 1, wherein said system is configured to measure an impedance across said balloon by means of said electric impedance measuring multimeter so as to measure the impedance that takes:
an elevated value reflecting an impedance of a vascular tissue while said balloon is inflated within a blood vessel to completely block a flow of blood;
a decreased value resulting from increased ion permeability of cell membranes after said vascular tissue is heated through an ablation of said balloon; and then
an elevated value again after said vascular tissue gets vaporized or carbonized.

8. (canceled)

9. The balloon catheter ablation system according to claim 1 further comprising a radiofrequency generator connectable across said bipolar electrode pair.

10. The balloon catheter ablation system according to claim 1 wherein said balloon is constituted using any one of: a cryoballoon; a laser balloon; an ultrasonic balloon; or a hot balloon employing a heating element or a nichrome wire as a heat source.
